(19) United States
(12) Patent Application Publication
(10) Pub. No.: US 2023/0235401 A1
(43) Pub. Date: Jul. 27, 2023

(54) BIOMARKERS FOR SMOKE EXPOSURE

(71) Applicant: Trustees of Boston University, Boston, MA (US)

(72) Inventors: Jerome S. Brody, Boston, MA (US); Avrum Spira, Newton, MA (US); Jennifer E. Beane-Ebel, Fort Collins, CO (US); Marc E. Lenburg, Brookline, MA (US)

(21) Appl. No.: 17/940,776

(22) Filed: Sep. 8, 2022

Related U.S. Application Data
(63) Continuation of application No. 15/336,469, filed on Oct. 27, 2016, now abandoned.
Application No. 15/336,469 is a continuation of application No. 14/584,960, filed on Dec. 29, 2014, now abandoned.
Application No. 14/584,960 is a continuation of application No. 13/346,444, filed on Jan. 9, 2012, now abandoned.
Application No. 13/346,444 is a continuation of application No. 12/905,897, filed on Oct. 15, 2010, now abandoned.
Application No. 12/905,897 is a continuation of application No. 12/234,368, filed on Sep. 19, 2008, now abandoned.
(60) Provisional application No. 60/994,637, filed on Sep. 19, 2007.

(51) Int. Cl. C12Q 1/6883

(52) U.S. Cl. CPC C12Q 1/6883; C12Q 1/6844; C12Q 2600/158

(57) ABSTRACT

Sensitive biomarker(s) to identify individuals with past exposure to tobacco smoke based on gene expression are disclosed. Such biomarkers may be used, for example, for epidemiological studies related to smoke exposure, to provide insights into the mechanisms leading to reversible and persistent effects of tobacco smoke that may explain former smokers’ increased risk for developing tobacco-induced lung disease, and/or to provide novel targets for chemoprophylaxis.

### CROSS REFERENCE TO RELATED APPLICATIONS

This application is a continuation of U.S. application Serial No. 15/336,469, filed Oct. 27, 2016, which is a continuation of U.S. application Serial No. 14/584,960, filed Dec. 29, 2014, which is a continuation of U.S. application Serial No. 13/346,444, filed Jan. 9, 2012, which is a continuation of U.S. application Serial No. 12/905,897, filed Oct. 15, 2010, which is a continuation of U.S. application Serial No. 12/234,368, filed on Sep. 19, 2008, which claims the benefit of U.S. provisional application Serial No. 60/994,637, filed Sep. 19, 2007, the entire teachings of which are incorporated herein by reference in their entirety.

### STATEMENT REGARDING FEDERALLY SPONSORED RESEARCH

This invention was made with government support under NIH/NCI CA106506 and NIH/NCI CA124640 awarded by The National Institute of Health. The government has certain rights in the invention.

### BACKGROUND OF THE INVENTION

Tobacco use remains the leading preventable cause of death in the United States, and cigarette smoking is the primary cause of chronic obstructive pulmonary disease (COPD) and respiratory-tract cancers. Smoking is responsible for approximately 440,000 deaths per year in the U.S. and results in $75 billion in direct medical costs and $82 billion in lost productivity (MMWR Morb Mortal Wkly Rep 2002, 51: 300-303). Exposure to tobacco smoke is widespread; approximately 45 million Americans are current smokers and 46 million are former smokers (MMWR Morb Mortal Wkly Rep 2005, 54: 509-513). The risk of dying from smoking-related diseases such as lung cancer and COPD remains elevated for former smokers compared to those who have never smoked (Halpern et al., J Natl Cancer Inst 1993, 85: 457-464). In the Dorn Study of U.S. veterans, the Kaiser Permanente Prospective Mortality Study, and American Cancer Society Cancer Prevention Study I (CPS-I) populations, the risk of death from lung cancer among former smokers was elevated above the risk for those who had never smoked 20 or more years following cessation (Shopland et al., Monograph No. 8(NIH Publ No 97-4213), 9-10. 1997. USDHHS, National Institutes of Health, National Cancer Institute. Ref Type: Serial (Book, Monograph)). As an increasing fraction of current smokers become former smokers, the absolute risk of lung cancer in the population will decline; however, an increasing number of lung cancer cases will occur among former smokers (Burns, Cancer 2000, 89: 2506-2509). It would therefore be useful to understand why former smokers remain at risk for lung cancer after smoking cessation in order to develop chemoprophylaxis treatments that could reduce risk.

Categorizing smoking-related changes in airway gene expression by their degree of reversibility upon smoking cessation would provide insights into the mechanisms leading to persistent gene expression changes in the airway epithelium exposed to tobacco smoke. Further understanding of these mechanisms may aid in understanding why former smokers remain at risk for developing lung cancer years of quitting smoking and perhaps aid in the development of treatments to lower this risk. In addition, biomarkers of past tobacco smoke exposure based on the expression of the genes that do not return to baseline levels after smoking cessation have the potential to provide useful tools for epidemiological and drug development studies.

### BRIEF SUMMARY OF THE INVENTION

One embodiment of the invention provides one or more biomarkers for detecting past exposure to tobacco smoke. The biomarker may comprise at least one gene with an expression pattern that is irreversibly or slowly reversibly altered by exposure to tobacco smoke. Such a gene may be, for example, any one or more of the following: TNFSF13, MUC5B, MT1F, MT1X, MT1G, FAM107A, CX3CL1, CCND2, SULF1, MMP10, PLA1A, ITM2A, PECI, MAOB, SLC6A16, CCDC33, PDGFC, TNS3, SEC14L3, HLF, TNS1, TMCC1, NQO1, PIR, GPX2, PLA2G10, TLE1, CEACAM6, TM4SF1, CEACAM5, SRPX2, UPK1B and SERPINB13. In one embodiment, the gene is irreversibly altered by exposure to tobacco smoke and may be, for example, one or more of CEACAM5 or NQO1. In another embodiment the gene is slowly reversibly altered by exposure to tobacco smoke and can be, for example, MT1X, TNFSF13, MT1G, CX3CL1, MT1F, FAM107A and combinations thereof.

In another embodiment, the invention provides methods for using biomarkers for detecting past exposure to tobacco smoke in a mammal. The method may include: (a) providing a biological sample, e.g., a biological sample from an airway passage of the mammal, wherein the biological sample comprises a gene expression product from at least one gene that has an expression pattern that is irreversibly or slowly reversibly altered by exposure to tobacco smoke, and (b) detecting the expression of said gene. For example, the gene may be one or more of the following: TNFSF13, MUC5B, MT1F, MT1X, MT1G, FAM107A, CX3CL1, CCND2, SULF1, MMP10, PLA1A, ITM2A, PECI, MAOB, SLC6A16, CCDC33, PDGFC, TNS3, SEC14L3, HLF, TNS1, TMCC1, NQO1, PIR, GPX2, PLA2G10, TLE1, CEACAM6, TM4SF1, CEACAM5, SRPX2, UPK1B and SERPINB13. In one embodiment, the gene is irreversibly altered by exposure to tobacco smoke and may be, for example, one or more of CEACAM5 or NQO1. In another embodiment the gene is slowly reversibly altered by exposure to tobacco smoke and can be, for example, MT1X, TNFSF13, MT1G, CX3CL1, MT1F, FAM107A and combinations thereof. The mammal can be, for example, a human. Biological samples may be provided, for example, from bronchial, nasal or buccal epithelium, and can be a cell sample or tissue sample, e.g., a biopsied tissue sample. In one embodiment, detection of gene expression is accomplished using an oligonucleotide array having immobilized thereon one or more nucleotide sequences or fragments or portions thereof which are probes for one or more genes which are irreversibly or slowly reversibly altered by exposure to tobacco smoke.

In another embodiment, the invention provides a method of using one or more biomarkers for characterizing a mammal’s response to tobacco smoke. The method may include: (a) providing a biological sample, e.g., a biological sample from an airway passage of the mammal, wherein the biological sample comprises a gene expression product (e.g., mRNA or protein) of at least one gene that has an expression pattern that is irreversibly or slowly reversibly altered by exposure to tobacco smoke, and (b) detecting expression of said gene, wherein expression of said gene(s) is correlated with the mammal’s response to tobacco smoke. For example, the gene may be one or more of the following: TNFSF13, MUC5B, MT1F, MT1X, MT1G, FAM107A, CX3CL1, CCND2, SULF1, MMP10, PLA1A, ITM2A, PECI, MAOB, SLC6A16, CCDC33, PDGFC, TNS3, SEC14L3, HLF, TNS1, TMCC1, NQO1, PIR, GPX2, PLA2G10, TLE1, CEACAM6, TM4SF1, CEACAM5, SRPX2, UPK1B and SERPINB13. In one embodiment, the gene is irreversibly altered by exposure to tobacco smoke and can be, for example, one or more of CEACAM5 or NQO1. In another embodiment the gene is slowly reversibly altered by exposure to tobacco smoke and can be, for example, MT1X, TNFSF13, MT1G, CX3CL1, MT1F, FAM107A and combinations thereof. The mammal can be, for example, a human. Biological samples may be provided, for example, from bronchial, nasal or buccal epithelium and can be a cell sample or tissue sample, e.g., a biopsy tissue sample. In one embodiment, detection of gene expression is accomplished using an oligonucleotide array having immobilized thereon one or more nucleotide sequences or fragments or portions thereof which are probes for one or more genes having an expression pattern which is irreversibly or slowly reversibly altered by exposure to tobacco smoke.

In another embodiment, the invention relates to an oligonucleotide array having immobilized thereon one or more probes for a biomarker gene disclosed herein. In particular embodiments one or more probes for more than one biomarker gene are immobilized on an oligonucleotide array. In preferred embodiments the array contains probes only for one or more biomarker genes disclosed herein, i.e., does not contain probes for other genes.

In another embodiment the invention provides methods for using the biomarkers described herein to screen candidate agents and/or assess treatment regimens. For example, expression of one or more biomarker genes disclosed herein can be sampled at various time points during treatment or before and after use of a candidate agent to identify regimens and agents which alter expression of said biomarker genes. Preferred regimens and agents will alter said biomarker gene expression such that the expression pattern of one or more genes which are slowly reversibly altered or irreversibly altered by exposure to tobacco smoke is similar to the expression pattern of said genes in nonsmokers. Treatment regimens and candidate agents which alter gene expression in this manner may be useful in the therapy and/or prophylaxis of lung disease associated with exposure to tobacco smoke.

Thus, in some embodiments the invention provides methods of assessing a candidate agent comprising treating (e.g., contacting) a cell (e.g., an isolated cell or a cell in a tissue or animal) with a candidate agent and comparing expression of one or more biomarker genes disclosed herein before and after treatment of said cell. A candidate agent which alters said biomarker gene expression such that the expression pattern of one or more genes which are slowly reversibly altered or irreversibly altered by exposure to tobacco smoke is similar to the expression pattern of said genes in nonsmokers is an agent which may be useful in the therapy and/or prophylaxis of lung disease associated with exposure to tobacco smoke.

The invention also provides methods of assessing a treatment regimen for efficacy comprising administering a treatment regimen to a cell, tissue or individual (e.g., patient) and assessing expression of one or more biomarker genes disclosed herein at multiple time points (e.g., two or more times before, during and/or after treatment). A treatment regimen which alters said biomarker gene expression such that the expression pattern of one or more genes which are slowly reversibly altered or irreversibly altered by exposure to tobacco smoke is similar to the expression pattern of said genes in nonsmokers is an efficacious regimen which may be useful in the therapy and/or prophylaxis of lung disease associated with exposure to tobacco smoke.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a flowchart depicting methodology for gene classification by degree of reversibility upon smoking cessation. For each probeset, the relationship between gene expression in log₂ scale (ge), age, current smoking status (x_{curr}), former smoking status (x_{form}), and the interaction between former smoking status and months elapsed since quitting smoke (x_{tq}) was examined with the linear regression model. Genes differentially expressed between current (C) and never (N) smokers were categorized based on their behavior in former smokers (F) relative to never smokers as a function of time since smoking cessation.

FIGS. 2A-2B are graphs depicting the characteristics of genes classified as irreversible, slowly reversible, or rapidly reversible based on their behavior in former smokers. FIG. 2A shows the numbers of genes up-regulated (red) or down-regulated (blue) in current smokers compared to never smokers. The percentage of genes up-regulated in smoking decreases from the most to the least reversible tertile of rapidly reversible genes and is lowest in the slowly reversible and irreversible genes. FIG. 2B shows the age-adjusted fold change between never versus former smokers (y-axis) plotted as a function of time since quitting smoking (x-axis) for the genes classified as slowly reversible. All the slowly reversible genes are down-regulated in smoking. The time point that the fold change equals 1.5 (see dotted line) is defined as the time that the genes become reversible. The time point at which this occurs is greater than 78 months (6.5 years) after smoking cessation for all of the slowly reversible genes.

FIGS. 3A-3B are a series of charts depicting the quantitative real time PCR results for select genes across never, former, and current smokers. For each graph sample identifiers for never (orange), former (purple), and current (green) smokers are listed along the x-axis. The sample identifications P1, P2, and P3 refer to three samples collected prospectively from never smokers that do not have corresponding microarrays. The months since smoking cessation are listed below each former smoker. The relative expression level on the y-axis is the ratio of the expression level of a particular sample versus that of a dummy reference sample. FIG. 3A shows plots of two rapidly reversible genes, CYP1B1 and ALDH3A1. FIG. 3B shows plots of two irreversible genes, CEACAM5 and NQO1.

FIG. 4 shows the relationship between samples according to the expression of genes with different reversibility characteristics. Principal component analyses (PCA) are shown on the left for the slowly reversible and irreversible genes (n=34) (A) and the most reversible rapidly reversible genes (n=46) (B). False-color heatmaps are shown on the right (C) for the slowly reversible and irreversible genes (top) and the most reversible tertile of rapidly reversible genes (bottom). Never, former, and current smokers are colored in orange, purple, and green respectively. The PCA and heatmaps were constructed using gene expression data normalized to a mean of zero and a standard deviation of 1. Never and current smokers are organized according to increasing age and former smokers are ordered by decreasing time since quitting smoking (denoted by the gradient) along the sample axis in the heatmap. Affymetrix identifications and HUGO gene symbols are listed for each gene as well as membership in two over-represented functional categories by EASE analysis.

FIGS. 5A-5B are charts depicting similarities and differences between the collected dataset and other bronchial airway datasets. FIG. 5A shows gene set enrichment analysis (GSEA) that was used to determine if there was a gene expression relationship between other airway datasets (see Table 2, below, for a description of Condition 1 and 2) and the collected dataset based on the genes identified to be regulated by smoking. The normalized enrichment score is plotted for datasets that had a false discovery rate (FDR) < 0.25. FIG. 5B shows gene lists derived from functional categories and chromosomal locations found to be over-represented by EASE analysis in the collected dataset were tested for enrichment in the collected dataset and the other 10 datasets using GSEA. A false-color heatmap of the positive (red) and negative (blue) normalized enrichment scores (with a FDR < 0.25) is shown for each category. An asterisk indicates the results passed a more strict FDR < 0.05. The nine datasets and conditions that yielded significant results in either (A) or (B) are indicated in Table 2 by the presence of a single asterisk.

### DETAILED DESCRIPTION OF THE INVENTION

As described herein, it has been discovered that exposure to smoke (e.g., tobacco smoke) produces alterations in gene expression which range from reversible alteration to slowly reversible alteration to irreversible alteration. Such alterations in expression have been identified herein for specific genes, resulting in gene expression profiles which can be used to identify and distinguish, for example, former smokers and never smokers. Perhaps more importantly these gene expression profiles and/or biomarkers can be used to guide treatment regimens and/or drug discovery efforts. For example, potentially effective treatment regimens and drug candidates may be identified by assessing their effect on the expression of genes identified herein. Treatment regimens and candidate agents which alter expression of one or more genes from an expression pattern correlated with smoking to or toward an expression pattern of said gene(s) correlated with non-smoking may be treatment regimens or candidate agents with efficacy in therapy or prophylaxis of diseases associated with smoking (e.g., lung cancer). In particular, treatment regimen and/or candidate agent effect on slowly reversible and/or irreversible genes may be assessed.

One embodiment of the invention provides one or more biomarkers which indicate a physical condition such as exposure to smoke, such as tobacco smoke. The biomarker may comprise, for example, one or more genes that are irreversibly, slowly reversibly, rapidly reversibly or reversibly affected by smoke. In a preferred embodiment, irreversibly altered genes may be used to construct a biomarker of past exposure to tobacco smoke capable of classifying an independent set of former and current smokers.

One embodiment of the invention provides methods of using the one or more biomarkers to identify an exposure state of an individual exposed to smoke. The individual may be a current smoker, former smoker, nonsmoker with secondhand smoke exposure or nonsmoker. In one embodiment, a biomarker of smoke exposure may provide a useful tool for epidemiologic studies. In another embodiment, the biomarkers may be used to identify individuals at risk of smoke-related disease by categorizing the degree of epithelial cell injury. The biomarkers may also be used to characterize an individual’s response to tobacco smoke exposure as acute or long-lasting.

One embodiment of the invention identifies genes for use in a biomarker by measuring gene expression in cells affected by smoke using an oligonucleotide microarray. In a preferred embodiment, gene expression in large airway epithelial cells is measured. Cells may be obtained from populations comprising individuals that have never smoked, currently smoke or formerly smoked. Genes may be classified according to their long-term response to smoke exposure, i.e., whether the effects of exposure are reversible, slowly reversible, rapidly reversible or irreversible. In one embodiment, statistical analysis is used to classify the genes.

As used herein, “airway passage” refers to air passages throughout the respiratory tree. Airway passages include, but are not limited to, bronchial, nasal and buccal passages.

As used herein, “altered by exposure to tobacco smoke” refers to gene expression that is altered from its baseline gene expression profile, for example in comparison to expression in a nonsmoker.

As used herein, “Additional data file 1” refers to a dataset for classifications of genes differentially expressed between current and never smokers according to their behavior in former smokers. For each gene the following information is given: the Affymetrix identification, the HUGO gene symbol, the direction of the change (up- or down- regulated in current smokers with respect to never smokers), the gene classification based on behavior of former smokers, and the percent reversibility.

As used herein, “Additional data file 2” refers to a summary of human bronchial epithelial datasets downloaded from GEO (Gene Expression Omnibus). For each dataset the following information is included: GEO series identification, microarray platform, cell type, where the cells were obtained, cell donor information (if applicable), number of samples, experiment type, exposure, experiment description, data preprocessing, and PUBMED identification (if applicable).

As used herein, “Additional data file 3” refers to a the Average Pearson correlations between 7 pairs of replicate samples where probeset gene expression values were determined using Microarray Suite 5.0 (MAS 5.0), log-transformed data from Microarray Suite 5.0 (Log₂ MAS 5.0), and Robust Multichip Average (RMA). The average, standard deviation, and median of the correlation coefficients are shown. GCRMA and RMA maximize the correlation between replicate samples.

As used herein, “Additional data file 4” refers to the GEO identifications for never, former, and current smokers. This file explains how the samples used in the present study overlap with previous publications. GEO identifications are provided for each sample for the present study and for the previously published studies (each study used different data preprocessing). GEO identification 1 refers to the study published in PNAS in 2004 (15210990), GEO identification 2 refers to the study published in Nature Medicine in 2007 (17334370), and GEO identification 3 refers to the present study. The study published in Nucleic Acids Research in 2005 (15608264) did not have an accompanying GEO submission.

As used herein, “Additional data file 5” refers to the Quantitative Real Time PCR Primer Sequences for the four candidate genes (ALDH3A1, CEACAM5, CYP1B1, and NQO1) designed with PRIMER EXPRESS software (Applied Biosystems), and the primer sequences of the housekeeping gene (GAPDH) adopted from “Accurate normalization of real-time quantitative RT-PCR data by geometric averaging of multiple internal control genes” by Vandesompele J. et al.

In an exemplary embodiment, airway epithelial cells were collected from a sample (n=104) of never, current, and former smokers and statistical models were developed to identify the gene expression changes associated with smoking. As used herein, this sample of airway epithelial cells is referred to as “the collected dataset”. Linear models may be used to identify genes differentially expressed in airway epithelium between never and current smokers and characterize their gene expression levels in former smokers that quit smoking at different time points. Thus, the degree to which these gene expression changes are reversible upon smoking cessation may be determined or categorized. The relationship between these gene expression changes and a number of publicly available human bronchial epithelial microarray datasets may be compared to provide insights into common mechanisms airway epithelial cells use in response to a variety of different toxins.

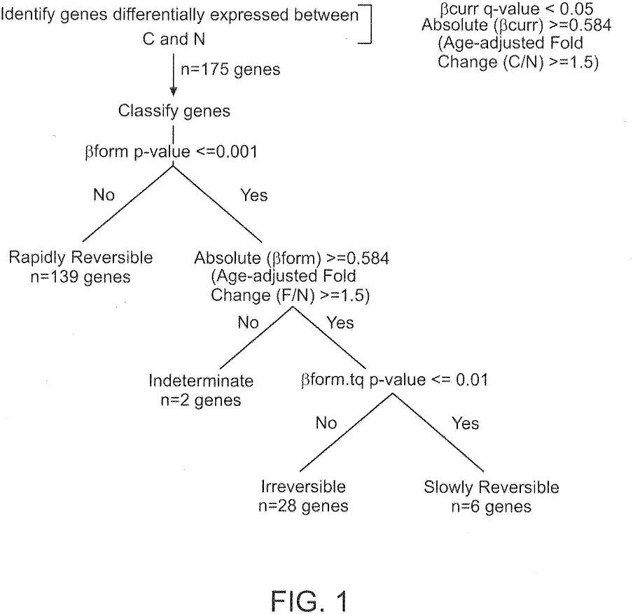

In one embodiment, changes in gene expression were measured using oligonucleotide microarrays. Linear models identified 175 genes differentially expressed between current and never smokers, and classified these as irreversible (n=28), slowly reversible (n=6), or rapidly reversible (n=139) based on their expression in former smokers. Referring to FIG. 1, genes were classified as Rapidly Reversible if there was not a significant difference between former and never smokers. Genes were classified as Indeterminate if there was a significant difference between former and never smokers, but the age-adjusted fold change between former and never smokers was not greater than or equal to 1.5. If the fold change criterion was met, genes were classified as Slowly Reversible if there was a significant relationship between gene expression and time since quitting smoking or as Irreversible if there was not a significant relationship between gene expression and time since quitting smoking. A greater percentage of irreversible and slowly reversible genes were down-regulated by smoking, suggesting possible mechanisms for persistent changes such as allelic loss at 16q13. Similarities with airway epithelium gene expression changes caused by other environmental exposures suggest that common mechanisms are involved in the response to tobacco smoke.

The majority (79%) of genes differentially expressed between current and never smokers are rapidly reversible upon smoking cessation while the rest are either slowly reversible or irreversible. Differences between the rapidly reversible and slowly reversible or irreversible genes further suggest that their expression might be regulated through distinct mechanisms. The rapidly reversible genes have different biological functions than the slowly reversible or irreversible genes, suggesting that they might distinguish between an acute response to tobacco smoke and a more long-lasting response to tobacco smoke-induced damage. The gene expression consequences of tobacco smoke exposure show significant commonalities with the gene expression changes observed in other human bronchial airway gene expression datasets involving tobacco smoke. Commonalities with human bronchial airway datasets involving other exposures suggest that the response to tobacco smoke exposure involves a number of common bronchial airway pathways.

In one embodiment it was found that many of the rapidly reversible genes are up-regulated by smoking and are involved in a protective or adaptive response to tobacco exposure and the detoxification of tobacco smoke components. The cytochrome p450s, CYP1A1 and CYP1B1, for example, are among the rapidly reversible genes and are involved in the oxidation of many compounds including fatty acids, steroids, and xenobiotics. CYP1A1 and CYP1B1 have been previously described as being up-regulated in response to smoke (Nagaraj et al., Toxicol Lett 2006, 165: 182-194) and CYP1B1 polymorphisms can influence the risk of developing lung cancer among never smokers (Wenzlaff et al., Carcinogenesis 2005, 26: 2207-2212). Several aldo-keto reductases, like AKR1B10 and AKR1C1, are also rapidly reversible upon smoking cessation. Aldo-keto reductases are soluble NADPH oxidoreductases that are involved in the activation of polycyclic aromatic hydrocarbons present in tobacco smoke and in the detoxification of highly carcinogenic NNK (nicotine-derived nitrosamino-ketone) compounds (Jin and Penning Annu Rev Pharmacol Toxicol 2007, 47: 263-292). Another class of rapidly reversible genes are the aldehyde dehydrogenases, such as ALDH3A1, that are involved in the oxidation of toxic aldehydes produced from oxidative stress and exposure to tobacco smoke (Vasiliou and Nebert, Hum Genomics 2005, 2: 138-143). Both the cytochrome p450s and the aldehyde dehydrogenases have been found to be up-regulated in respiratory tissue from rats exposed to smoke (Gebel et al., Carcinogenesis 2004, 25: 169-178) and the aldo-keto reductases are up-regulated in normal bronchial epithelium and non-small cell lung tumor tissue from smokers compared with non-smokers (Woenckhaus et al., J Pathol 2006, 210: 192-204).

The genes listed above as well as most of the differentially expressed genes that are members of the GO (Gene Ontology) molecular function category, oxidoreductase activity, are among the most highly reversible genes, suggesting that the up-regulation of these genes is driven by the acute exposure to smoke-related toxins and returns to baseline soon after the exposure to these compounds ceases. The induction of these genes in airway epithelial cells after 15 minutes of exposure to tobacco smoke (GSE2302) lends further support to this hypothesis.

In another embodiment, the slowly reversible and irreversible genes reflect a more permanent host-response to tobacco smoke. Interestingly, several have been associated with the development of cancers of epithelial origin. CEACAM5, carcinoembryonic antigen-related cell adhesion molecule 5, is irreversibly up-regulated by smoking and has been shown to be over-expressed in the serum of cancer patients with lung adenocarcinoma (Hotta et al., Anticancer Res 2000, 20: 2177-2180) and colorectal cancer (Goldstein and Mitchell, Cancer Invest 2005, 23: 338-351). SULF1 (sulfatase 1), a gene irreversibly down-regulated by smoking influences the sulfation state of residues present on heparin sulfate proteoglycans (HSPGs). HSPGs are involved in cell adhesion and mediate growth factor signaling. SULF1 was found to be down-regulated in ovarian, breast, pancreatic, renal cells, and hepatocellular carcinoma cell lines (Lai et al., J Biol Chem 2003, 278: 23107-23117) and head and neck squamous carcinomas (Lai et al., Oncogene 2004, 23: 1439-1447). UPK1B, uroplakin 1B, plays a role in strengthening and stabilizing the apical cell surface through interactions with the cytoskeleton (Yu et al., J Cell Biol 1994, 125: 171-182). UPK1B is irreversibly down-regulated by smoking and has been shown to be reduced or absent in bladder carcinomas through CpG methylation of the proximal promoter. (Varga et al., Neoplasia 2004, 6: 128-135; Cowled et al., Neoplasia 2005, 7: 1091-1103).

The enrichment of down-regulated genes among the irreversible, slowly reversible, and the least rapidly reversible genes may suggest that genetic or epigenetic mechanisms such as chromosomal loss (Wistuba et al., J Natl Cancer Inst 1997, 89: 1366-1373; Powell et al., Clin Cancer Res 1999, 5: 2025-2034) or changes to promoter methylation status (Wistuba et al., Oncogene 2002, 21: 7298-7306; Guo et al., Clin Cancer Res 2004, 10: 5131-5136) might account for these gene expression differences. Given the relatively rapid turnover of airway epithelial cells, the persistence of these changes post-smoking cessation may result from a clonal growth advantage to epithelial cells in the airway harboring these changes. Several of the down-regulated slowly reversible genes are present in cytoband 16q13, where a number of metallothioneins are located. Metallothioneins have the ability to bind both essential metals like copper and iron as well as toxic metals such as cadmium and mercury. They also have detoxification and antioxidant properties and may be involved in cell proliferation and differentiation (Cherian et al., Mutat Res 2003, 533: 201-209). MT3 has been shown to be down-regulated by hypermethylation in non small cell lung tumors and cell lines (Zhong et al., Oncogene 2007, 26(18): 2621-2634). In addition, metallothioneins are thought to regulate some zinc-dependent transcription factors such as the tumor suppressor p53 by donating zinc (Meplan et al., Oncogene 2000, 19: 5227-5236). Potential loss or methylation of the chromosomal locus containing several metallothionein genes may impair the ability of epithelial cells to protect or to repair cellular injury from future environmental exposures that occur after smoking cessation.

In another embodiment, the observed effect of smoking and smoking cessation described above was confirmed by comparing the collected dataset with other publicly available human bronchial epithelial cell datasets involving a variety of exposures. In general, reproducibility of findings using different microarray datasets across similar experimental conditions and cell types has not traditionally been common practice because overlap between differentially expressed gene sets is often surprisingly small (Evsikov and Solter, Science 2003, 302: 393). New methodologies for comparing datasets make the task more feasible (Subramanian et al., Proc Natl Acad Sci USA 2005, 102: 15545-15550), and provide more powerful methods for determining commonalities between the observed responses of a particular cell type under one or more conditions. The tobacco exposure-associated gene expression changes observed were concordant in three other datasets involving tobacco smoke exposures. The most significant similarity involved the gene expression consequences of tobacco smoke exposure in the small airway epithelium of never and current smokers (GSE3320). This suggests that the field of injury in response to tobacco smoke is similar throughout both the large and small airways. In one embodiment, significant similarity was found between genes that were up-regulated by smoking and the immediate gene expression changes resulting from acute tobacco exposure (GSE2302). This similarity was significant for both rapidly reversible and irreversible/slowly reversible up-regulated genes. The lack of similarity among genes down-regulated by smoking in the collected dataset and GSE2302 may reflect differences between acute and chronic cigarette smoke exposure, and suggests that up- and down-regulated irreversible gene expression may occur through different biological mechanisms.

In another embodiment, there were significant similarities between genes up- and down-regulated by smoking and the gene expression differences in additional datasets such as GSE5264 (cells undergoing mucociliary differentiation) and GSE1815 (interferon gamma-treated cells). These may provide biological insights about the nature of airway epithelial response to tobacco smoke exposure. The gene expression program that accompanies mucociliary differentiation has led to the hypothesis that cultured “undifferentiated” epithelial cells may more closely resemble damaged epithelium or neoplastic lesions in vivo because many genes associated with normal squamous epithelia, squamous cell carcinomas, or EGFR signaling are more highly expressed in undifferentiated cells. (Ross et al., Am J Respir Cell Mol Biol 2007). The similarity between genes up-regulated by smoking in the collected dataset and genes that are more highly expressed early in mucociliary differentiation together with the similarity between genes down-regulated by smoking in the collected dataset and genes that are more highly expressed late in mucociliary differentiation might therefore reflect the cellular damage induced by smoke exposure. In addition, there was similarity between genes up-regulated by smoking in the collected dataset and genes down-regulated by treatment with interferon gamma. As interferon gamma plays a role in lung inflammatory responses, these similarities suggest that tobacco smoke exposure may suppress inflammatory responses in the airway. The relationships described above and presented in the results between the collected dataset and the other datasets are confirmed at a pathway level and may suggest that oxidoreductase activity and electron transporter activity are among the important molecular functions of the bronchial epithelium that are regulated in response to a wide range of carcinogenic, inflammatory, and toxic exposures.

In another embodiment, a biomarker of tobacco smoke exposure was developed as an additional validation of the gene changes observed in response to smoking and smoking cessation. Using genes irreversibly altered by cigarette smoke, an independent sample set of former and current smokers (GSE4115) and a sample set of smokers and non-smokers (GSE5372) was classified with high accuracy. These preliminary biomarker results demonstrate the potential for developing a useful epidemiological tool if the gene expression biomarker could be ultimately extended to less invasive sites such as the buccal and nasal epithelium as these are tissues that are also directly exposed to tobacco smoke. Biomarkers of exposure are frequently used to improve upon or validate information about tobacco smoke exposure obtained by questionnaire; however, current biomarkers of tobacco exposure (e.g. cotinine (SRNT Subcommittee on Biochemical Verification: Biochemical verification of tobacco use and cessation. Nicotine Tob Res 2002, 4: 149-159) and NNAL, a metabolite of the tobacco-specific nitrosamine NNK (Hecht et al., Cancer Res 1999, 59: 590-596; Hecht et al., J Natl Cancer Inst 2004, 96: 107-115) are limited to detecting recent exposure. Development of a biomarker for long-term past exposure using gene expression could have widespread epidemiological utility.

In another embodiment, the invention relates to an oligonucleotide array having immobilized thereon one or more probes for a biomarker gene disclosed herein. In particular embodiments one or more probes for more than one biomarker gene are immobilized on an oligonucleotide array. In preferred embodiments the array contains probes only for one or more biomarker genes disclosed herein, i.e., does not contain probes for other genes. Methods for making oligonucleotide arrays are known in the art. Arrays described herein may be used for prognostic and screening purposes to facilitate gene expression assessments as described herein.

In another embodiment the invention provides methods for using the biomarkers described herein to screen candidate agents and/or assess treatment regimens. For example, expression of one or more biomarker genes disclosed herein can be sampled at various time points during treatment or before and after use of a candidate agent to identify regimens and agents which alter expression of said biomarker genes. Preferred regimens and agents will alter said biomarker gene expression such that the expression pattern of one or more genes which are slowly reversibly altered or irreversibly altered by exposure to tobacco smoke is similar to the expression pattern of said genes in nonsmokers. Treatment regimens and candidate agents which alter gene expression in this manner may be useful in the therapy and/or prophylaxis of lung disease associated with exposure to tobacco smoke.

Thus, in some embodiments the invention provides methods of assessing a candidate agent comprising treating (e.g., contacting) a cell (e.g., an isolated cell or a cell in a tissue or animal) with a candidate agent and comparing expression of one or more biomarker genes disclosed herein before and after treatment of said cell. A candidate agent which alters said biomarker gene expression such that the expression pattern of one or more genes which are slowly reversibly altered or irreversibly altered by exposure to tobacco smoke is similar to the expression pattern of said genes in nonsmokers is an agent which may be useful in the therapy and/or prophylaxis of lung disease associated with exposure to tobacco smoke. Candidate agents may be, for example, known therapeutic agents or agents without previously identified therapeutic, prognostic or diagnostic effect and include but are not limited to small molecules and biological agents.

The invention also provides methods of assessing a treatment regimen for efficacy comprising administering a treatment regimen to a cell, tissue or individual (e.g., patient) and assessing expression of one or more biomarker genes disclosed herein at multiple time points (e.g., two or more times before, during and/or after treatment). A treatment regimen which alters said biomarker gene expression such that the expression pattern of one or more genes which are slowly reversibly altered or irreversibly altered by exposure to tobacco smoke is similar to the expression pattern of said genes in nonsmokers is an efficacious regimen which may be useful in the therapy and/or prophylaxis of lung disease associated with exposure to tobacco smoke. Treatment regimens may include but are not limited to treatment with an agent which is administered to a cell, tissue or individual (e.g., drug treatment or herbal treatment) as well as treatments such as chemotherapy, radiation, etc. Treatment regimens may utilize one agent/regimen or combinations of multiple agents and regimens.

The practice of the present invention will employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F.M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, NY (jointly referred to herein as “Harlow and Lane”), and Beaucage et al. eds., Current Protocols in Nucleic Acid Chemistry John Wiley & Sons, Inc., New York, 2000).

### Exemplary Embodiment

### Materials and Methods

To obtain the collected dataset, airway epithelial brushings were obtained from never, current, and former smokers undergoing fiberoptic bronchoscopy between April 2003 and January 2006 (n= 281 samples, including replicates (n=12)). Subjects with lung cancer or unknown lung cancer status were excluded from the analyses (n=119) given the previously described airway-wide field of injury occurring in smokers with lung cancer. Demographics including age, pack years, and months since quitting smoking were obtained from each subject. The subjects were recruited from four institutions: Boston University Medical Center, Boston, MA; Boston Veterans Administration, West Roxbury, MA; Lahey Clinic, Burlington, MA; and St. James’s Hospital, Dublin, Ireland. The Institutional Review Boards of all of the medical centers approved the study and all subjects provide written informed consent. With the exception of 9 samples, all samples used in the analyses were included in studies previously published (Spira et al., Proc Natl Acad Sci U S A 2004, 101: 10143-10148; Spira et al., Nat Med 2007; Shah et al., Nucleic Acids Res 2005, 33: D573-D579) (Additional data file 4).

Demographic information for the 21 never, 31 former, and 52 current smokers used in the present study are shown in Table 1. There were significant differences in age for all possible two-way comparisons by t-test among the three groups (P < 0.05 by pairwise t-tests), however, there was no significant difference between cumulative tobacco exposure between the former and current smokers.

TABLE 1

| | Never | Former | Current |
|---|---|---|---|
| n | 21 | 31 | 52 |
| Age | 32.3 (10.7) | 55.9 (14.7) | 48.6 (15.2) |
| Pack years | | 34.0 (30.1) | 34.5 (34.2) |
| Months Since Quitting | | 145.2 (162.82) | |

### Airway Epithelial Cell Collection

Bronchial airway epithelial cells were obtained from the right mainstem bronchus with an endoscopic cytobrush (Cellebrity Endoscopic Cytobrush, Boston Scientific, Boston, MA). RNA was isolated and its integrity and epithelial cell content was confirmed as described previously in the art (Spira et al., Nat Med 2007, 13(3); 361-366).

### Microarray Data Acquisition

6-8 µg of total RNA was processed, labeled and hybridized to Affymetrix HG-U133A GeneChips containing 22,283 probesets as described previously in the art (Spira et al., Proc Natl Acad Sci USA 2004, 101: 10143-10148). Log₂-normalized probe-level data were obtained using the GCRMA algorithm (Wu, The Journal of the American Statistical Association 2004, 99: 909-917) because it maximized the correlation between technical replicates compared to the Microarray Suite 5.0 algorithm and performed equivalently to a similar method, RMA (Robust Multichip Average) (Irizarry et al., Biostatistics 2003, 4: 249-264)(Additional data file 3). All 281 samples (including replicates) collected during the study period were used for sample filtering. A z-score filter was applied to filter out arrays of poor quality. The filter involves computing an average z-score statistic across all probesets for each sample using z-score normalized data so that the mean gene expression value across all samples for each probeset is 0 and the standard deviation is 1. (Spira et al., Nat Med 2007, 13(3); 361-366). Samples with high average z-scores were eliminated in addition to the 119 samples with lung cancer or unknown lung cancer status leaving 104 samples - 21 never smokers without cancer (N), 31 former smokers without cancer (F), and 52 current smokers without cancer (C). The data can be accessed through GEO accession GSE7895.

### Modeling the Effect of Smoking and Smoking Cessation

Linear regression models were used to identify genes differentially expressed as a function of tobacco smoke exposure. These genes were further analyzed to describe gene expression changes upon smoking cessation. For each probeset, the relationship between gene expression in log₂ scale (ge), age, current smoking status (x_{curr}=1 for current smokers and 0 otherwise), former smoking status (x_{form} =1 for former smokers and 0 otherwise), and the interaction between former smoking status and months elapsed since quitting smoke (x_{tq}) was examined with the linear regression model:

$$\begin{matrix}g {e}_{i} = {β}_{o} + {β}_{a g e} ∗ {x}_{a g e} + {β}_{a g e} ∗ {x}_{c u r r} + {β}_{f o r m} ∗ {x}_{f o r m} + {β}_{f o r m . t q} ∗ {x}_{f o r m} ∗ \\ {x}_{t o} + {E}_{s}\end{matrix}$$

where εᵢ represents the error assumed to be normally distributed. The equation describes the expression of a probe i for never and current smokers as:

$$N e v e r S m o k e r : g {e}_{i} = {β}_{o} + {β}_{a g e} ∗ {x}_{a g e} + {e}_{l}$$

$$C u r r e n t S m o k e r : g {e}_{i} = {β}_{o} + {β}_{a g e} ∗ {x}_{a g e} + {β}_{c u r r} ∗ 1 + {e}_{i}$$

Age was included in the model to control for the potentially confounding effect of age and smoking status (Table 1). By difference, the age-adjusted fold change between current and never smokers is 2^β_{curr}. The standard least-square method was used to estimate the regression coefficients, and the significance of the regression coefficients was tested using the t-test. Goodness of fit of the models was assessed by analysis of residuals.

Probesets differentially expressed between current and never smokers were defined by two requirements. 1) A q-value (Storey and Tibshirani, Proc Natl Acad Sci U S A 2003, 100: 9440-9445) for the regression coefficient β_{curr} < 0.05 (which corresponded to P < 7.6*10-4). The q-value is the expected proportion of false positives incurred when calling probesets with this q-value or smaller significant and was used to correct for the multiple comparisons. 2) An absolute value of the β_{curr} coefficient > 0.584, which corresponds to an age-adjusted fold change of expression > 1.5. A fold change cutoff was chosen because of the little power provided by the sample size to detect smaller changes using multivariate linear regression models. (Sebastiani, Bayesian Analysis 2006, 1: 707-732). After the q-value and fold change criteria were applied, probesets with the same gene symbol (according the June 2006 HG-U133A Affymetrix annotation files), were filtered such that only the probeset with the lowest q-value was retained. All probesets without gene symbol annotation, however, were included.

The behavior of the probesets selected in the first comparison was further analyzed in former smokers. The linear model shown in Equation 1 describes the expression of a probe i in former smokers as:

$$\begin{matrix}F o r m e r s m o k e r : g {e}_{i} = {β}_{o} + {β}_{a g e} ∗ {x}_{a g e} + {β}_{f o r m} ∗ 1 + {β}_{f o r m . t q} ∗ 1 ∗ \\ {x}_{t q} + {e}_{i}\end{matrix}$$

and allows further classification of probes based on the pattern of expression in former smokers as a function of time since quitting smoking with respect to never smokers (See FIG. 1). Equation 4 shows that the expression of a probeset in a former smoker differs from that of a never smoker if the regression coefficient β_{form} is significantly different from 0. The difference can be unrelated to time elapsed since quitting if the regression coefficient β_{form.tq} is not significantly different from 0, or it can change over time if β_{form.tq} is significantly different from 0. In the latter case, when the changes over time are monotone, the time point at which the fold change was equal to 1.5 (|β_{form} + β_{form.tq} * x_{form} * x_{tq}| = 0.584) can be identified. This led to the following definitions: 1) A gene was defined as Rapidly Reversible if the regression coefficient β_{form} was not significantly different from 0 (P≥0.001); 2) A gene was defined as Irreversible if the regression coefficient β_{form.tq} was not significantly different from 0 (P≥0.01), but the β_{form} coefficient was significantly different from 0 (P<0.001) and the absolute β_{form} coefficient was > 0.584 (corresponding to an age-adjusted fold change between formers and never smokers > 1.5); 3) A gene was defined as Indeterminate if the regression coefficient β_{form.tq} was not significantly different from 0 (P≥0.01), but the β_{form} coefficient was significantly different from 0 (P<0.001) and the absolute β_{form} coefficient ≤ 0.584; 4) A gene was defined as Slowly Reversible if the regression coefficients β_{form} and β_{form.tq} were significantly different from 0 (P<0.001, and P<0.01, respectively) and the absolute β_{form} coefficient > 0.584. The genes were characterized by the time point (tq) where |β_{form} + β_{form.tq} * x_{form} * x_{tq}| = 0.584. This corresponds to the time point where the age-adjusted fold change of never versus former smokers was equal to 1.5 (since all genes classified as slowly reversible were down-regulated by smoking).

In addition, to characterize the range of reversibility among genes designated as rapidly reversible, the percent reversibility for each gene was calculated according to the formula:

$$1 - \frac{2 \left|{β}_{f o r m}\right|}{2 \left|{β}_{c u r r}\right|} .$$

In rare cases, where the former smoker versus never smoker fold change was slightly higher than the current versus never smoker fold change, the percentage was set to 100%; and in cases where the former smokers expression levels returned to a slightly lower level than never smokers the percentage was set at 0%. The reversible genes were divided into tertiles based on this reversibility percentage.

### Relationship of Irreversible and Reversible Genes to Other Bronchial Epithelial Cell Datasets

NCBI’s microarray data repository, GEO (Edgar et al., Nucleic Acids Res 2002, 30: 207-210), was queried for human bronchial epithelial cell samples in August 2006. Processed data was downloaded from GEO for each dataset (10 datasets total) that contained more than 3 total samples, contained more than 2 total samples per condition, and that was processed using whole genome arrays (Additional data file 2). The 175 genes differentially expressed between current and never smokers were mapped to the various datasets. Principal component analyses (PCA) were performed for each dataset across the mapped probesets using z-score normalized data. Graphs of the first versus second principal component were used as guides to decide what groups of samples show differential expression of the genes identified as being differentially expressed between current and never smokers.

The relationship was subsequently defined quantitatively using Gene Set Enrichment Analysis (GSEA (Subramanian et al., Proc Natl Acad Sci U S A 2005, 102: 15545-15550) (available through the GenePattern software (Reich et al., Nat Genet 2006, 38: 500-501). The samples in each dataset from above were divided into two groups based on the experimental design -- control versus the treated samples. If the samples were treated at two different time points, however, the time points were either combined into one treated group or kept separate for different comparisons between the control and the treated group at a particular time point (the PCA analyses from above were used to guide these decisions).

TABLE 2

| Dataset | Condition 1 | Condition 2 | # Samples in Condition 1 | # Samples in Condition 2 | Significant Dataset |
|---|---|---|---|---|---|
| GSE3320 | Non smokers | Smokers | 5 | 6 | * |
| GSE2302 | Control | Smoke 15 min, 24 hr recovery | 9 | 5 | ** |
| GSE2302 | Control | Smoke 15 min,4 and 24 hr recover | 9 | 9 | * |
| GSE2302 | Control | Smoke 15 min, 4 hr recovery | 9 | 4 | |
| GSE1276 | Untreated,2 and 4 hrs S9+CSCA/CSCB | 8 and 12 hrs S9+CSCA/CSCB | 10 | 8 | |
| GSE1276 | S9 2, 4, 8 and 12 hrs | S9+CSCA 2, 4, 8, 12 hrs | 8 | 8 | |
| GSE1276 | S9 2,4, 8and 12 hrs | S9+CSCB 2, 4, 8, 12 hrs | 8 | 8 | |
| GSE1815 | Untreated 8 and 24 hrs | INF-gamma treated 24 hrs | 9 | 5 | ** |
| GSE1815 | Untreated 8 and 24 hrs | INF-gamma treated 8 and 24 hrs | 9 | 9 | * |
| GSE2111 | Control | Zinc Sulfate | 4 | 4 | * |
| GSE2111 | Control | Vanadium | 4 | 4 | |
| GSE5264 | Days 0 through 8 | Days 10 through 28 | 14 | 16 | * |
| GSE620 | Control | 4-PBA 12 and 24 hrs | 5 | 6 | * |
| GSE620 | Control | 4-PBA 24 hrs | 5 | 3 | ** |
| GSE3397 | Control | RSV 24 hrs | 4 | 4 | |
| GSE3397 | Control | RSV 4 and 24 hrs | 4 | 8 | * |
| GSE3183 | Control | IL13 4, 12, and 24 hrs | 6 | 9 | |
| GSE3183 | Control | IL13 24 hrs | 6 | 3 | |
| GSE3183 | Control +IL13 4 hrs | IL13 12 and 24 hrs | 9 | 6 | * |
| GSE3004 | Pre allergen challenge | Post allergen challenge | 5 | 5 | |

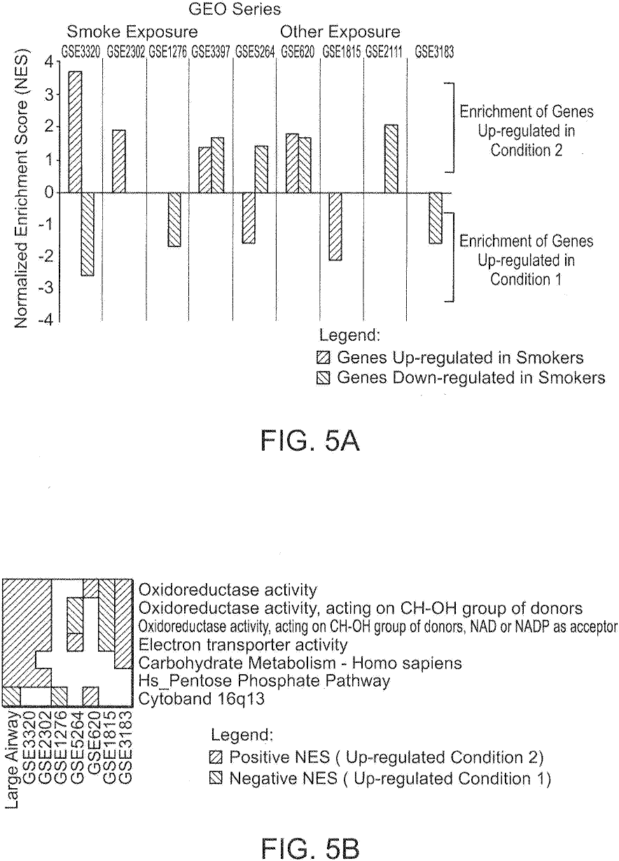

Table 2 shows the two group comparisons examined for each of the GEO datasets. The GEO series accessions as well as the description and numbers samples in each of the conditions are listed for each comparison. Datasets where differentially expressed genes between condition 1 and condition 2 demonstrated similarity to differentially expressed genes between current and never smokers in the collected dataset are indicated by the presence of one or two asterisks. Only comparisons indicated by a single asterisk are shown in FIG. 5.

For each comparison, the probesets were mapped to gene symbols using GSEA’s Affymetrix annotation files; or, in the case of the two non-Affymetrix arrays (datasets GSE2302 and GSE1276), the annotation file human-library.txt available at http://omrf.ouhsc.edu/~frank/human-library.txt was used. The file provides the following annotation for each probeset on the arrays: the name of the gene being interrogated, the description of the gene, and the Genbank identification. The redundant gene symbols were collapsed using a script written in the R Language for Statistical Computing (R Development Core Team: R: A language and environment for statistical computing. Vienna, Austria: R Foundation for Statistical Computing; 2005) that retained the probesets with the highest absolute signal to noise ratio. This strategy was chosen so that all potentially differentially expressed genes were included in the analyses. The collapsed datasets were evaluated using GSEA to determine if the gene sets listed below were also differentially expressed in the datasets by the signal to noise statistic comparing treatment versus control. The following gene sets were tested: 1) slowly reversible and irreversible genes up-regulated by smoking; 2) slowly reversible and irreversible genes down-regulated by smoking; 3) rapidly reversible genes up-regulated by smoking; 4) rapidly reversible genes down-regulated by smoking; 5) all genes up-regulated by smoking; 6) all genes down-regulated by smoking. Significant enrichment was defined as a p-value<0.05 and a false discovery rate < 0.25 derived using 10,000 gene-label permutations.

### Identifying Common Biological Themes Across Datasets

EASE (Hosack et al., Genome Biol 2003, 4: R70) was used to identify Gene Ontology Molecular Function categories, KEGG pathways, GenMAPP pathways, and chromosomal cytobands over-represented among genes designated as slowly reversible and irreversible or reversible compared to all annotated genes on the Affymetrix U133A microarray (Permutation P <= 0.01). GSEA was subsequently performed using gene lists derived from each significant EASE category to identify which of these over-represented categories were enriched in genes up- or down-regulated in each GEO dataset (Table 2, above). The enrichment of EASE categories observed in the collected dataset was confirmed using GSEA in which the β_{curr} smoking status coefficient (representing the magnitude of the difference between current and never smokers) was used to order the probesets.

### Biomarker for Past Smoke Exposure

A biomarker of past exposure using the irreversible genes (n=28) was trained on the never and former smokers using a support vector machine (SVM) classification system with a linear kernel via the R package e1071, which includes functions for latent class analysis, short time Fourier transform, fuzzy clustering, support vector machines, shortest path computation, bagged clustering, naive Bayes classifier (The e1071 Package. http://cran.r-project.org/src/contrib/Descriptions/e1071.html 2006, Ref Type: Generic) The SVM model was tested on the training set and three different test sets - the current smokers in the present study, current and former smokers that were not included in the present study from dataset GSE4511, and GSE5372 which included gene expression measurement from large airway epithelial cells in 4 non-smokers and 5 current smokers at different time points (22 samples total) (Heguy et al., Physiol Genomics 2007, 29: 139-148). The biomarker was used to predict the class of the GSE5372 samples taken at the initial time point (n=9). P-values for the performance of the biomarker were established by randomizing the class labels of the training set, re-running the algorithm 1000 times, and calculating the proportion of the random runs that produced biomarkers that had the same or better accuracy in the test set samples.

### Quantitative Real Time PCR

Quantitative RT-PCR analysis was used to confirm the differential expression of a two irreversible and two rapidly reversible genes known to play roles in the detoxification of tobacco smoke and pathogenesis of lung cancer. Primer sequences for the four genes (ALDH3A1, CEACAM5, CYP1B1, and NQO1) were designed with PRIMER EXPRESS software (Applied Biosystems) (Additional data file 5). Primer sequences of the housekeeping gene (GAPDH) were adopted from Vandesompele (Vandesompele et al., Genome Biol 2002, 3: RESEARCH0034). RNA samples (1 µg of residual RNA from the samples used in the microarray analysis) were treated with DNAfree (Ambion), according to the manufacturer’s protocol, to remove contaminating genomic DNA. Total RNA was reverse-transcribed by using random hexamers (Applied Biosystems) and SuperScript II reverse transcriptase (Invitrogen). The resulting first-strand cDNA was diluted with nuclease-free water (Ambion) to 4 ng/µl. PCR amplification mixtures (25 µl) contained 20 ng template cDNA, 12.5 µl of 2X SYBR Green PCR master mix (Applied Biosystems) and 300 nM forward and reverse primers. Forty cycles of amplification and data acquisition were carried out in an ABI Prism 7700 Sequence Detector (Applied Biosystems). Threshold determinations were automatically performed by Sequence Detection Software (version 1.9.1) (Applied Biosystems) for each reaction. All real-time PCR experiments were carried out in triplicate on each sample (mean of the triplicate shown). Four never, 3 former, and 2 current smokers were chosen for each gene based on the amount of RNA available (17 samples total: 6 current, 7 former, and 1 never smoker from this study and 3 additional never smokers collected prospectively).

### Statistical Analysis

Statistical analyses and hierarchical clustering may be conducted using R statistical software v 2.2.1 and Bioconductor packages (Gentleman et al., Genome Biol 2004, 5:R80) .

### Effect of Smoking and Smoking Cessation

Three-hundred and forty-three probesets show significant differences in intensity between current and never smokers based on the significance of the current smoking status variable in the linear model (q-value < 0.05 corresponding to a P < 7.6*10⁻⁴). Two-hundred and nineteen probesets remained after applying a filter to retain only probesets where the absolute current smoking status coefficient was greater than or equal to 0.584 (corresponds to an age-adjusted fold change between current and never smokers of 1.5). Finally, after filtering out redundant probesets (probesets representing the same gene) from this set of 219 probesets, probesets representing 175 genes remained. There was a high degree of overlap (78%) between genes that had been previously identified as being perturbed by active cigarette smoke exposure (Spira et al., Proc Natl Acad Sci USA 2004, 101: 10143-10148) and the 175 genes identified by the linear model.

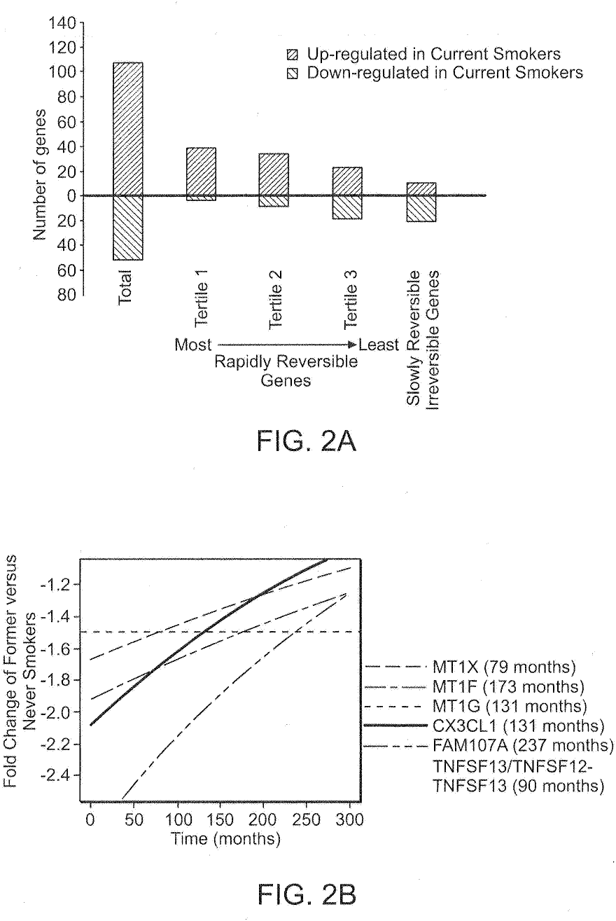

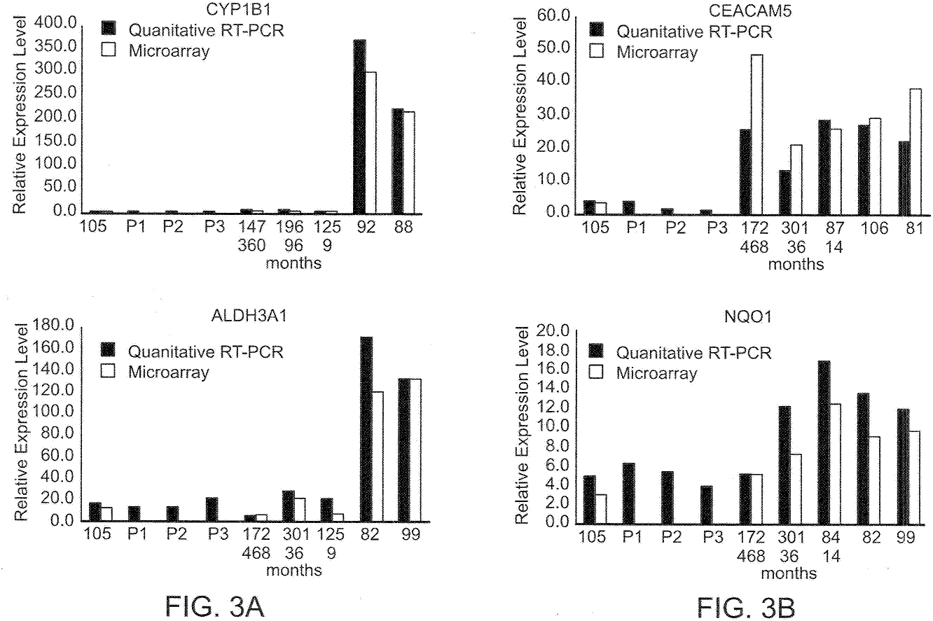

The 175 genes differentially expressed between current and never smokers were classified as irreversible, slowly reversible, or rapidly reversible based on their behavior in former smokers (FIG. 1). This yielded 28 irreversible genes, 6 slowly reversible genes, 139 rapidly reversible genes, and 2 indeterminate genes. The 139 rapidly reversible genes were subsequently divided into three equal tertiles based on their percent reversibility (see FIG. 2A). Genes classified as slowly reversible were characterized by the time point at which the age-adjusted fold change between never and former smokers dropped below the threshold of 1.5. The time point is greater than 78 months for all of the genes classified as slowly reversible (FIG. 2B). A list of the 175 genes as well as their reversibility classification and percentage is displayed in Additional data file 1. The gene expression of 2 irreversible and 2 rapidly reversible genes was confirmed by quantitative real time PCR (FIG. 3).

Interestingly, 65% of the slowly reversible and irreversible genes were down-regulated by smoking, while only 23% of rapidly reversible genes were down-regulated by smoking (Fisher exact test P = 7.2*10⁻⁶). Amongst the rapidly reversible genes, those that were down-regulated tended to be the least reversible as determined by percent reversibility (Fisher exact test P=0.0001 comparing the proportion of down-regulated genes in each fertile). Genes down-regulated by smoking, for example, account for only 6.5% of the most reversible tertile of rapidly reversible genes (n=46), but account for 43% of the least reversible tertile (FIG. 2A).

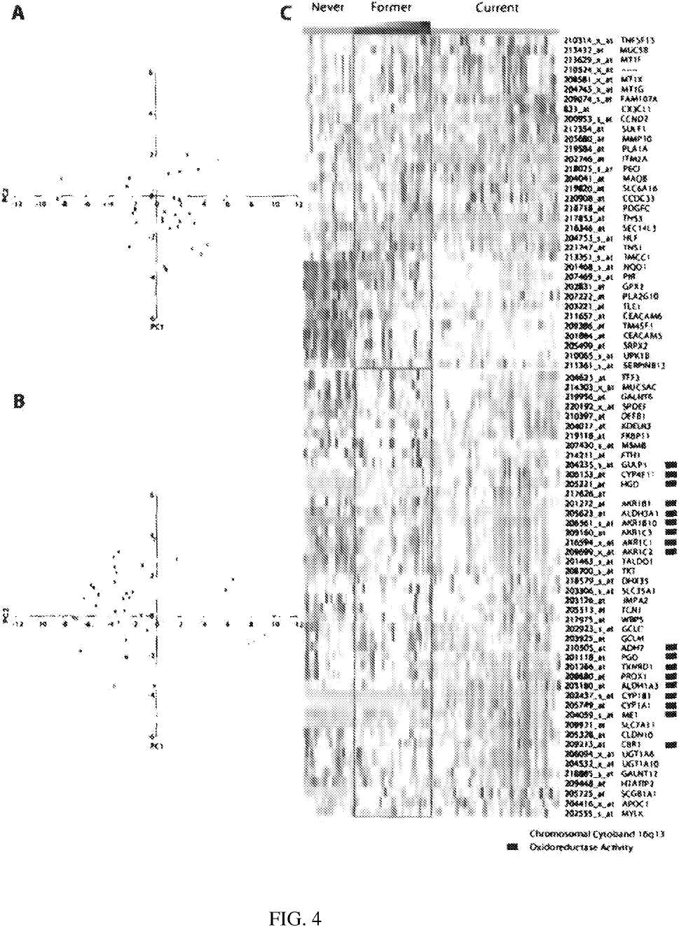

A principal component analysis (PCA) shows that former smokers are similar to current smokers according to the expression of the 34 irreversible and slowly reversible genes (FIG. 4A), while a PCA using the most reversible tertile of rapidly reversible genes demonstrates the reverse (FIG. 4B). The PCA analyses also demonstrate heterogeneity among former smokers. There are 3 former smokers (time since quit smoking 96, 156, and 300 months) in FIG. 4A that cluster with the never smokers and 3 former smokers (time since quit smoking 3, 6, and 14 months) in FIG. 4B that cluster with the current smokers, raising the possibility that these individuals may have a different physiological response to tobacco smoke. A heatmap of the gene expression levels of never, former, and current smokers across the slowly reversible and irreversible genes as well as the most reversible tertile of rapidly reversible genes demonstrates the greater proportion of genes down-regulated by smoking among the irreversible and slowly reversible genes (FIG. 4C) .

EASE (Hosack et al., Genome Biol 2003, 4:R70) was used to identify which Gene Ontology Molecular Function categories (Harris et al., Nucleic Acids Res 2004, 32: D258-D261), KEGG pathways (Kanehisa et al., Nucleic Acids Res 2002, 30: 42-46), GenMAPP pathways (Dahlquist et al., Nat Genet 2002, 31: 19-20), and chromosomal cytobands are over-represented (Permutation P <= 0.01) among genes designated as irreversible and slowly reversible or reversible compared to all annotated genes on the Affymetrix U133A microarray as shown in Table 3.

TABLE 3

| | | | | |
|---|---|---|---|---|
| System | Category | EASE score | Permutation P-value | Reversibility Group |
| GO Molecular Function | oxidoreductase activity | 8.49E-08 | 1.00E-03 | Rapidly Reversible Genes |
| GO Molecular Function | electron transporter activity | 4.60E-06 | 1.00E-03 | Rapidly Reversible Genes |
| GenMAPP Pathway | Hs Pentose Phosphate Pathway | 8.59E-06 | 1.00E-03 | Rapidly Reversible Genes |
| GO Molecular Function | oxidoreductase activity, acting on the CH-OH group of donors, NAD or NADP as acceptor | 5.73E-05 | 2.00E-03 | Rapidly Reversible Genes |
| GO Molecular Function | oxidoreductase activity, acting on CH-OH group of donors | 7.59E-05 | 2.00E-03 | Rapidly Reversible Genes |
| KEGG Pathway | Carbohydrate Metabolism - Homo sapiens | 1.71E-04 | 4.00E-03 | Rapidly Reversible Genes |
| Chromosomal Location | 16q13 | 2.02E-03 | 1.00E-03 | Slowly Reversible and Irreversible Genes |

The metallothioneins (MT1G, MT1X, MT1F) and the chemokine CX3CL1 are located on Cytoband 16q13, which is over-represented among irreversible and slowly reversible genes (FIG. 4A). Although not all metallothioneins in the region of 16q13 were present in the list of 175 genes, all of the probesets on the U133A corresponding to MT4, MT3, MT2A, MTLE, MT1M, MT1F, MT1G, MT1H, and MT1X were down-regulated in current smokers. Genes involved in the metabolism of the carcinogenic components of cigarette smoke including electron transporter activity and oxidoreductase activity are over-represented among the rapidly reversible genes. Genes with oxidoreductase activity such as the aldo-keto reductases, aldehyde dehydrogenases, and the cytochrome p450s were predominantly present in the most reversible tertile of the rapidly reversible genes (Fisher Exact P= 1.3*10⁻⁵ comparing the proportions of genes in each tertile with oxidoreductase activity) (FIG. 4C).

### Enrichment of Irreversible and Reversible Genes in Bronchial Epithelial Cell Datasets

In order to confirm the impact of smoking on airway epithelial cell gene expression and examine the specificity of this response, the findings disclosed herein were compared with 10 other previously published human bronchial airway epithelial cell microarray datasets involving a variety of exposures (Additional data file 2). Principal component analyses (PCA) were performed for each of the 10 datasets across the 175 genes (differentially expressed between never and current smokers) that could be mapped to the microarray platform used in each study using gene symbols. One-hundred seventy-three out of the 175 genes had gene symbols, and all of these mapped to the following datasets: GSE5264, GSE3397, GSE3320 GSE3183, GSE2111, and GSE620. One-hundred forty-nine genes mapped to GSE2302 and GSE1276, and 135 genes mapped to datasets GSE1815 and GSE3004. The relationship between the experimental conditions studied in each of the GEO datasets to the collected dataset was defined using Gene Set Enrichment Analysis (GSEA) (Table 3, above). Significant GSEA results (p-value < 0.05 and false discovery rate < 0.25) are displayed in FIG. 5A. Genes that are perturbed by smoking in the present study are also enriched or differentially expressed (by the signal to noise metric (Golub et al., Science 1999, 286: 531-537) in the three smoking datasets corroborating the gene expression changes identified by the linear model. Genes up- and down-regulated by smoking in the collected dataset were most closely related to (had the highest enrichment scores) genes differentially expressed in dataset GSE3320. GSE3320 was generated using epithelial cells obtained from the small airways (10^{th} to 12^{th} order) at bronchoscopy from both non-smoking and smoking volunteers, and is thus the mostly closely related to the collected dataset (Hackett et al., Am J Respir Cell Mol Biol 2003, 29: 331-343). Genes up-regulated by smoking in the collected dataset are also up-regulated in dataset GSE2302. The lack of enrichment in genes down-regulated by smoking in the collected dataset and genes down-regulated in GSE2302 may reflect differences between the effects of acute and chronic cigarette smoke exposure. The collected dataset is likely to capture the gene expression consequences of chronic exposure while bronchial cell cultures in the GSE2302 series were only exposed to smoke for 15 minutes and assayed at 4 and 24 hour time points after the exposure.

In contrast to the above two datasets, the similarity between the gene expression changes in the collected dataset and those in GSE1276 was not as strong. GSE1276 used bronchial epithelial cells obtained from cadavers to study the effects of the S9 microsomal fraction from 1254-Aroclor treated rats and cigarette smoke condensate from two different brands of cigarettes at 2, 4, 8, and 12 hour time points smoke condensate (Jorgensen et al., Cell Cycle 2004, 3: 1154-1168). Genes down-regulated by smoking in the collected dataset were also down-regulated in epithelial cells treated with S9 plus cigarette smoke condensate for 8 and 12 hours compared to earlier time points. The uniqueness of GSE1276 is potentially due to the S9 treatment which had unexpected broad effects on gene expression that may enhance or suppress the effects of the tobacco smoke condensate (Jorgensen et al., Cell Cycle 2004, 3: 1154-1168).

Genes that are perturbed by tobacco smoke exposure in the collected dataset also show some evidence of differential expression in six out of seven additional bronchial epithelial cell datasets. Genes up-regulated by smoking tended to be genes that are down-regulated by interferon gamma treatment for 24 hours in (GSE1815 (Pawliczak et al., Physiol Genomics 2005, 23: 28-45) suggesting that smoking may have an immunosuppressive effect; and also tended to be genes that are down-regulated at later time points during mucociliary differentiation (GSE5264 (Ross et al., Am J Respir Cell Mol Biol 2007)), suggesting that the damage caused by tobacco-smoke induces genes that are expressed more highly in undifferentiated epithelial cells. Genes down-regulated by smoking tended to be genes that are up-regulated in response to zinc sulfate (GSE2111 (Li et al., Environ Health Perspect 2005, 113: 1747-1754). These included the metallothionein genes (MT1X, MT1F, and MT1G). Taken together, the above results suggest that the bronchial epithelial cell response to tobacco smoke exposure consists of components that are shared with the response to a variety of other exposures.

### Identifying Common Biological Themes Across Datasets

In order to build upon the relationships between the datasets described above, additional relationships at the functional or pathway level were established. Gene lists composed of the genes in each of the over-represented gene categories (see Table 3, above) were used to determine if these gene categories tended to be differentially expressed in the other bronchial cell datasets using GSEA (FIG. 5B). This analysis shows that genes in five of the six functional categories that are induced by smoking and rapidly reversible upon smoking cessation also tended to be differentially expressed in two of the three smoking datasets. This further strengthens the notion that a similar bronchial epithelial response to tobacco smoke exposure is being detected in these datasets. Additionally, genes involved in oxidoreductase activity (which were found to be induced by smoking and rapidly reversible upon smoking cessation) are enriched among genes down-regulated during differentiation (GSE5264) or in response to interferon gamma treatment (GSE1815). These genes are also enriched among genes up-regulated in response to 4-PBA (GSE620) or IL-13 (GSE3183).

### Biomarker of Past Exposure

Irreversible changes in gene expression in response to tobacco smoke exposure suggest that a gene expression biomarker indicating whether an individual has ever been exposed to tobacco smoke can be developed. The ability of such a biomarker to accurately classify additional former smoker samples would serve as an important validation of the irreversible gene expression changes identified. A biomarker of tobacco exposure was constructed using the 28 irreversible genes and a training set of never and former smokers from the collected dataset (n=52). A support vector machine (SVM) classifier was able to classify 100% of the training set samples correctly. The SVM was then first used to predict the tobacco exposure status of the current smokers in the collected dataset. Not surprisingly, as these samples were used to define the 28 irreversible genes despite having not used these samples to develop the SVM, the SVM correctly predicted 89% of current smokers as having had exposure to cigarette smoke. The 6 current smokers predicted incorrectly had low pack-years (average was 9.5 in contrast to the group average of 34.5). In addition, current and former smokers from a previous study (GSE4115 (Spira et al., Nat Med 2007, 13(3); 361-366) that did not overlap with the samples used in this study were used as an additional test set. In this dataset, the SVM correctly classified 100% of current smokers and 81% of former smokers. Dividing the former smokers from dataset GSE4115 into 3 groups, former smokers who quit less than 2 years ago (n=12), former smokers who quit greater than or equal to 2 years but less than 10 years ago (n=15), and former smokers who quit greater than or equal to 10 years ago (n=20) yielded similar accuracies (83%, 80%, 80%, respectively). Finally, the SVM correctly predicted the class of all samples from non-smokers (n=4) and 80% of samples from smokers (n=5) from a recently published dataset (GSE5372). The accuracy of the biomarker in predicting samples from datasets GSE4115 and GSE5372 was significantly better than the accuracies obtained in 1000 runs that trained the SVM on class-randomized training sets (P=0.01 and P=0.001, respectively) (Table 4).

TABLE 4

| | Training Set | | | Test Set | GSE4115 | | | GSE5372 | | |
|---|---|---|---|---|---|---|---|---|---|---|
| | Never | Former | All | Current | Former | Current | All | Non-smokers | Smokers | All |
| # Classified Correctly | 21 | 31 | 52 | 46 | 38 | 38 | 76 | 4 | 4 | 8 |
| # Total Samples | 21 | 31 | 52 | 52 | 47 | 38 | 85 | 4 | 5 | 9 |
| Accuracy | 100.0% | 100.0% | 100.0% | 88.5% | 80.9% 80.9% | 100.0% | 89.4% | 100.0% | 80.0% | 88.9% |
| Mean Random Accuracy | | | 52.5% | 59.2% | | | 59.2% | | | 50.1% |
| P-value | | | 0 | 0.102 | | | 0.013 | | | 0.001 |

Table 4 shows a biomarker of tobacco smoke exposure constructed using the 28 irreversible genes. The accuracy of the biomarker is reported for the training set samples, test set samples, samples from dataset GSE4115 that do not overlap with the present study, and samples from GSE5372. The P-values represent the proportion of 1000 random training sets that have the same or better accuracy on the tested samples as the actual biomarker.

While some specific embodiments of the subject inventions are explicitly disclosed herein, the above specification is illustrative and not restrictive. The full scope of the inventions should be determined by reference to the claims, along with their full scope of equivalents, and the specification.

1-17. (canceled)

18. A method of determining if a human subject is a current or former smoker, comprising:
a) providing a biological sample from the human subject comprising airway epithelium, wherein the biological sample comprises gene expression products of a plurality of genes comprising one or more of MT1F, MT1X, MT1G, SULF1, TNFSF13, MUC5B, FAM107A, CX3CL1, CCND2, MMP10, PLA1A, ITM2A, PECI, MAOB, SLCA16, CCDC33, PDGFC, TNS3, SEC14L3, HLF, TNS1, NQO1, PIR, GPX2, PLA2G10, TLE1, CEACAM6, TM4SF1, CEACAM5, SRPX2, CYP1A1, CYP1B1, AKR1B10, AKR1C1, ALDH3A1 or UPK1B;
b) detecting the gene expression products of the plurality of genes in the biological sample and generating an expression dataset for the biological sample;
c) processing the expression dataset using a machine learning classifier to evaluate an expression pattern of the plurality of genes to classify the plurality of genes as irreversibly altered by exposure to smoke, slowly reversible after cessation of smoke exposure, or rapidly reversible after cessation of smoke exposure.

19. The method of claim 18, wherein the airway epithelium comprises bronchial, nasal, or buccal epithelium.

20. The method of claim 19, wherein the airway epithelium comprises bronchial epithelium.

21. The method of claim 18, wherein the plurality of genes comprises MT1F, MT1X, MT1G, SULF1, TNFSF13, MUC5B, FAM107A, CX3CL1, CCND2, MMP10, PLA1A, ITM2A, PECI, MAOB, SLCA16, CCDC33, PDGFC, TNS3, SEC14L3, HLF, TNS1, NQO1, PIR, GPX2, PLA2G10, TLE1, CEACAM6, TM4SF1, CEACAM5, SRPX2, CYP1A1, CYP1B1, AKR1B10, AKR1C1, ALDH3A1 and UPK1B.

22. The method of claim 18, wherein CEACAM5, SULF1 or NQO1 are classified as irreversibly altered by exposure to smoke.

23. The method of claim 22, wherein SULF1 is classified as irreversibly down-regulated by exposure to smoke.

24. The method of claim 22, wherein CEACAM5 is classified as irreversibly upregulated by exposure to smoke.

25. The method of claim 18, wherein CYP1A1, CYP1B1, AKR1B10, AKR1C1, or ALDH3A1 are classified as rapidly reversible after cessation of smoke exposure.

26. The method of claim 18, wherein the processing comprises determining that the human subject is a current or former smoker when the expression pattern of the plurality of genes is altered as compared to an expression pattern of the plurality of genes in an airway epithelium sample from a human that has never smoked.

27. The method of claim 18, wherein processing further comprises classifying the plurality of genes as (i) slowly reversible and irreversible genes up-regulated by smoking; (ii) slowly reversible and irreversible genes down-regulated by smoking; (iii) rapidly reversible genes up-regulated by smoking; or (iv) rapidly reversible genes down-regulated by smoking.

28. The method of claim 18, wherein the machine learning classifier is a support vector machine classifier.

29. The method of claim 18, wherein the machine learning classifier comprises a linear regression model.

30. The method of claim 29, wherein the linear regression model comprises the following equation: geᵢ = β₀ + β_{age} * x_{age} + β_{curr} * x_{curr} + β_{form} * x_{form} + β_{form} _{tq} * x_{form} * x_{tq} + εᵢ
wherein x_{curr} = 1 for current smokers and x_{curr} = 0 for others; and
wherein x_{form} = 1for former smokers and x_{form} = 0 for others.

31. The method of claim 30, wherein a gene is classified as rapidly reversible if the regression coefficient β_{form} is equal to zero with a p-value greater than or equal to 0.001.

32. The method of claim 30, wherein a gene is classified as irreversible if the regression coefficient β_{form} _{tq} is equal to zero with a p-value greater than or equal to 0.01. and the regression coefficient β_{form} is greater than 0.584.

33. The method of claim 30, wherein a gene is classified as indeterminate if the regression coefficient β_{form} _{tq} is equal to zero with a p-value greater than or equal to 0.01 and the regression coefficient β_{form} is less than or equal to 0.584.

34. The method of claim 30, wherein a gene is classified as slowly reversible if the regression coefficient β_{form} is greater than 0.584 and the regression coefficient β_{form} _{tq} is not equal to zero with a p-value less than 0.01.

35. A method of determining if a human subject is a current or former smoker, comprising:
a) providing a biological sample from the human subject comprising airway epithelium; and
b) assaying the biological sample for the expression of one or more genes selected from MT1F, MT1X, MT1G, SULF1, TNFSF13, MUC5B, FAM107A, CX3CL1, CCND2, MMP10, PLA1A, ITM2A, PECI, MAOB, SLCA16, CCDC33, PDGFC, TNS3, SEC14L3, HLF, TNS1, NQO1, PIR, GPX2, PLA2G10, TLE1, CEACAM6, TM4SF1, CEACAM5, SRPX2, CYP1A1, CYP1B1, AKR1B10, AKR1C1, ALDH3A1 or UPK1B.

36. The method of claim 35, wherein (b) further comprises assaying the biological sample for the expression of five or more genes selected from MT1F, MT1X, MT1G, SULF1, TNFSF13, MUC5B, FAM107A, CX3CL1, CCND2, MMP10, PLA1A, ITM2A, PECI, MAOB, SLCA16, CCDC33, PDGFC, TNS3, SEC14L3, HLF, TNS1, NQO1, PIR, GPX2, PLA2G10, TLE1, CEACAM6, TM4SF1, CEACAM5, SRPX2, CYP1A1, CYP1B1, AKR1B10, AKR1C1, ALDH3A1 or UPK1B.

37. The method of claim 35, wherein (b) comprises assaying the biological sample for the expression of MT1F, MT1X, MT1G, SULF1, TNFSF13, MUC5B, FAM107A, CX3CL1, CCND2, MMP10, PLA1A, ITM2A, PECI, MAOB, SLCA16, CCDC33, PDGFC, TNS3, SEC14L3, HLF, TNS1, NQO1, PIR, GPX2, PLA2G10, TLE1, CEACAM6, TM4SF1, CEACAM5, SRPX2, CYP1A1, CYP1B1, AKR1B10, AKR1C1, ALDH3A1 and UPK1B.
